(19) United States
(12) Patent Application Publication
(10) Pub. No.: US 2008/0294138 A1
(43) Pub. Date: Nov. 27, 2008

(54) Absorbent Product

(75) Inventors: Patrik Andersson, Billdal (SE); Maria Bogren, Askim (SE); Carin Hakansson, Billdal (SE); Kent Hermansson, Vastra Frolunda (SE); Linda Fransson, Romelanda (SE)

Correspondence Address: DRINKER BIDDLE & REATH (DC), 1500 K STREET, N.W., SUITE 1100, WASHINGTON, DC, 20005-1209, US

(73) Assignee: SCA Hygiene Products AB, Goteborg (SE)

(21) Appl. No.: 12/097,721

(22) PCT Filed: Dec. 16, 2005
(86) PCT No.: PCT/SE2005/001960

§ 371 Date: Jun. 16, 2008

(51) Int. Cl. A61F 13/15

(52) U.S. Cl. 604/385.23; 604/385.01

(57) ABSTRACT

Absorbent product including a liquid permeable perforated top sheet facing the wearer during use, a liquid impermeable back sheet facing away from the wearer during use, whereby longitudinally extending textile-like edges are positioned on both longitudinal sides of the top sheet, so that a central part of the top sheet is not covered by the textile-like edges, whereby the longitudinal diameter of the apertures of the top sheet is in the interval from 0.1 to 3.2 mm, preferably from 0.5 to 3.2 mm, more preferably from 0.9 to 2.4 and most preferably from 1.6 to 2.4 mm, wherein at least 70% of the area of the central part of the top sheet is apertured, and wherein the top sheet further includes perforations, that are positioned between the apertures of the top sheet.

### CROSS-REFERENCE TO PRIOR APPLICATION

This application claims priority to Application No. PCT/SE2005/001960 filed Dec. 16, 2005, which is incorporated by reference herein.

### FIELD OF THE INVENTION

The invention refers to an absorbent product, especially an incontinence protection, a sanitary napkin or a panty liner comprising a liquid permeable apertured top sheet facing the wearer, a liquid impermeable back sheet facing away from the wearer, and an absorbent structure positioned between the top sheet and the back sheet, and optionally a liquid distribution layer between the top sheet and the absorbent structure, wherein the top sheet and the back sheet are sealed together at the periphery of the product thereby forming an edge sealing, and whereby longitudinally extending textile-like edges are positioned on both longitudinal sides of the top sheet.

### BACKGROUND OF THE INVENTION

In order to provide an efficient liquid inlet in absorbent products, the characteristics of the top sheet material are important. Material choice and hydrophilic/hydrophobic character are examples of parameters that are important to optimize for the specific product and its specific purpose, i.e., if the product is intended to absorb large or small amounts of liquid, and the character of the liquid that is absorbed (blood, urine or a combination). One further aspect of the top sheet that is important for the function is the structure of the top sheet material, i.e., if it includes apertures, perforations, embossments and other structural characteristics, in order to e.g., provide efficient uptake and reduced risk for leakage.

EP-A-983758 discloses an absorbent article comprising a nonwoven top sheet, which is apertured in the central area. The open area is about 10% and the apertures have an effective size of at least 0.2 mm².

EP-A-761190 discloses an absorbent product such as a sanitary napkin having perforations of varying sizes for beneficial liquid uptake.

EP-A-523683 discloses an absorbent product such as a pantiliner comprising a top sheet (e.g., a perforated film) that is covered with a skin-friendly nonwoven material at its longitudinal edges. The open area of the perforated central part of the top sheet is 30-60%.

The present invention is concerned with absorbent products, whereby the demand for optimized absorbent capacity is high. It is important that the product has an efficient liquid intake, and it is important that the risk for leakage is minimized. Further, it is important that the product can function well at varying situations; it must be capable to take care of very large amounts of liquid at a short time period, as well as only a few droplets over a long period of time. Some consumers will use a product of this type each day, and some will use it only a few days every month. In order to satisfy these varying needs the product needs to have a quick liquid inlet and it needs to have a dry surface. A dry surface is very important for consumers wearing the product, e.g., during an entire working day. Hence, the product must have a low rewetting; i.e., the possibility for liquid that has been absorbed by the absorbent structure of the product to return to the top sheet must be low.

The absorbent products that are available today for this consumer category have shown to have non-satisfying rewetting properties.

Moreover, by using a large open area in a top sheet material, a quick liquid inlet is achieved. However, normally this will lead to non-satisfying rewetting properties, since liquid easily returns to the surface of the top sheet, especially when the product experiences pressure, which frequently is the case when the product is worn by the consumer, e.g., when the consumer moves or sits down.

Thus, many aspects must be considered and many demands must be met in order to provide a well-functioning product for this consumer category. Known products and prior art references address some of these demands, but it has shown to be difficult to meet all these demands in a single product. Further, since the product in some cases can be small in size, it is even further difficult to meet the demands by a single product.

The object of the invention is to provide an absorbent product that combines the relevant features discussed above in a single product, so that consumer demands with regard to liquid inlet, dry top sheet surface during use and wearer comfort are met.

### SUMMARY

The inventors of the present invention have succeeded in providing an absorbent product that solves these problems by a unique design of perforations and apertures of the top sheet. Thus, in a first aspect the invention refers to an absorbent product, having an apertured top sheet, wherein the longitudinal diameter of the apertures of the top sheet are in the interval from 0.1 to 6.0 mm, preferably in the interval from 0.1 to 3.2 mm, preferably from 0.5 to 3.2 mm, more preferably from 0.9 to 2.4 mm and most preferably from 1.6 to 2.4 mm, wherein at least 20%, preferably at least 50% and more preferably at least 70% of the width of the upper surface of the top sheet of the crotch part is apertured and/or perforated.

In a preferred embodiment, the top sheet further includes perforations that are positioned between the apertures of the top sheet.

Hereby, since relatively large apertures are used, liquid is quickly let through the top sheet to the absorbent structure. Further, since small perforations can be positioned between the apertures and the top sheet, at least in the crotch area, to a large extent is open, the acquisition properties are enhanced. Preferably, at least 80% of the width of the top sheet of the crotch part is apertured and/or perforated. Also, the apertures of the textile-like edges further improve the acquisition characteristics of the product, and decreases the risk for any leakage. Thus, the combination of apertures and perforations in the top sheet and in the textile-like edges, gives a product having outstanding qualities with regard acquisition among other qualities.

In a preferred embodiment, the top sheet is three-dimensionally formed, wherein each aperture has an upper longitudinal diameter (D) and a lower longitudinal diameter (d), wherein D is positioned closer to the top surface of the product, whereby D≧d. Preferably, D>d so that the apertures of the top sheet are basically funnel-shaped. Hereby an efficient inlet is achieved, and liquid that has been absorbed does not easily return to the surface of the top sheet as a result of the design of the apertures.

In a preferred embodiment the density of apertures in the central part of the top sheet (i.e., the part that is not covered by the textile-like edges) are in the interval from 3-15, preferably 6-12 and more preferably 7-9 apertures/cm². Thus, since the apertures are large, and the density of apertures is relatively low, the top sheet surface becomes relatively uneven and will be experienced by the users as soft and absorbing, thus enhancing wearer comfort. Also, it is preferred that the width of the textile edges is in the interval from 10-30%, preferably 18-22% of the total product width at the least wide position of the crotch part of the product. All these preferred features contribute to a balance that provides a product having quick liquid inlet and a dry surface.

In another preferred embodiment, the density of perforations (i.e., in this context small apertures) in the top sheet is in the interval of 20-500, preferably 70-250 and more preferably 120-170 perforations/cm².

Further, since the textile-like edges are preferably apertured, the risk for leakage is even further reduced, since any liquid that is not absorbed through the central part of the product will be absorbed through the textile-like edges. Thus, a liquid-absorbing “reservoir” is provided by the apertured textile-like edges.

Preferably, the ratio between the diameter of the apertures of the top sheet and the diameter of the apertures of the textile-like edges is at least 2. It is also preferred that the ratio between the open area of the top sheet and the open area of the textile-like edges is at least 2. Hence, by having larger apertures in the central part of the top sheet and/or a higher open area ratio in the central part of the top sheet, the absorbent capacity is higher where it is needed the most.

One important aspect of the invention is that the product of the invention has a high absorption capacity in relation to its size. In order to achieve this advantage, some properties should be shown by the product.

To start with, the liquid inlet materials (top sheet and liquid distribution layer) should be able to take care of a relatively large volume of liquid. This is achieved by using a top sheet material having funnel-shaped three-dimensional apertures, and by providing a bulky material, such as a high loft layer, beneath the top sheet. The three-dimensional structure of the apertured material together with the high loft layer gives a high free volume (void volume) that quickly can take care of the liquid. The funnel-shaped apertures will also “collect” the liquid initially so that it does not flow off the surface. The film of the top sheet material can also be pre-perforated with small apertures, so that the liquid also partly can be taken in between the large apertures.

Secondly, the inlet materials should be able to keep the liquid that has been taken up by the product during use. This is achieved by using the film of the top sheet material as a barrier against rewetting, i.e., to make it difficult for the liquid to return to the skin of the wearer. The super absorbent polymer (SAP) of the absorbent core has also, of course, a major contribution to keeping the absorbed liquid in the product. The three-dimensional structure of the top sheet and liquid distribution layer, preferably high loft layer, also makes the distance between the skin of the wearer and the core of the product longer, so that the product is experienced as drier.

Moreover, as a third point, the inlet materials need to provide a dry surface for the comfort of the wearer. This is achieved by ultra-sonically welding the top sheet material and the liquid distribution layer. Hereby, the top sheet material and liquid distribution layer can be properly drained.

Further, as a fourth point, the inlet materials should be soft and airy for the comfort of the wearer. This is achieved by that the material for the textile edges is made in a soft nonwoven for providing good comfort, and by that it includes apertures for being “airier” and for giving a good inlet.

Thus, the invention is further directed to an absorbent product wherein the product has at least one of the following characteristics (in accordance with the experimental results shown in the example section): (i) an acquisition time for a first inlet of 5 ml synthetic urine that is below 6 s, preferably equal to or less than 5 s, and an acquisition time for a second inlet of 5 ml synthetic urine that is below 8 s, preferably equal to or below 4 s; (ii) a DORUP (retention) value for a dosage of 7 ml synthetic urine that is below 1 g, preferably below 0.5 g and more preferably equal to or below 0.2 g; or (iii) a Rothwell (Absorption capacity) value of more than 45 g, preferably more than 55 g.

### BRIEF DESCRIPTION OF THE DRAWINGS

FIG. 1 illustrates a top plan view a panty liner having asymmetric shape of the invention including apertured textile-like edges.

FIG. 2 illustrates a top plan view another panty liner of the invention comprising apertured textile-like edges.

FIG. 3 illustrates a cross-sectional view of various layers of an absorbent product of the invention.

FIG. 4 is a principal drawing showing the top sheet structure and the liquid distribution layer. Specifically, the general structure of the funnel-shaped apertures of the top sheet is shown.

FIGS. 5-7 show experimental diagrams referring to the Example section of the invention.

### DETAILED DESCRIPTION OF THE PREFERRED EMBODIMENTS

The present invention pertains to an absorbent product. By an “absorbent product” is meant a product such as an incontinence protection, a sanitary napkin and a panty liner. The following definitions are provided below for further clarity regarding features of the present application.

By “open area” is meant the percentage of the surface of a material that is composed of apertures and perforations. In the case of presence of apertures that are three-dimensional, thus having e.g., a larger diameter at the upper edge compared to the lower edge, the open area thus refers to the effective open area, and thus the (smaller) lower diameter.

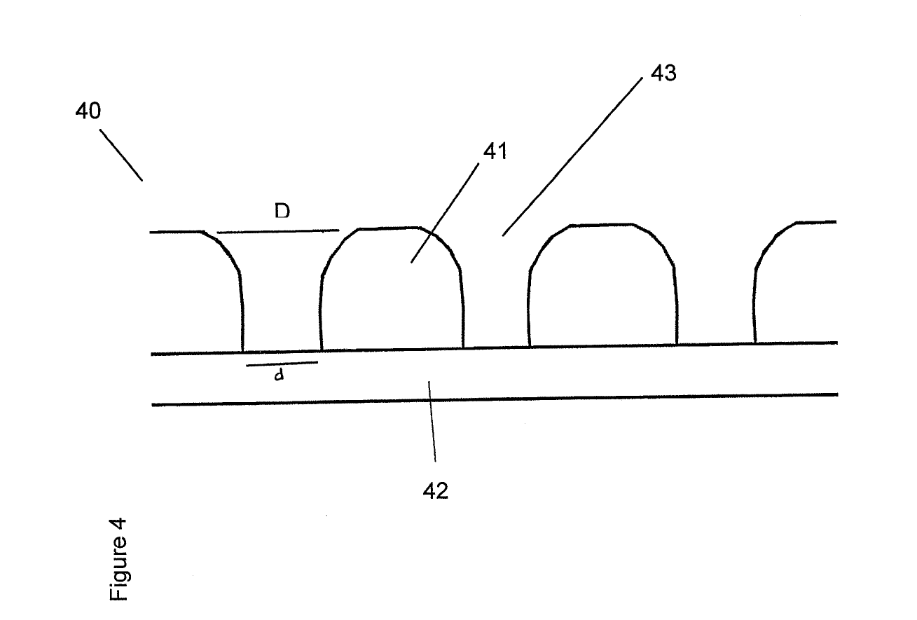

By “at least x % of the width of the upper surface top sheet of the crotch part being apertured and/or perforated” is meant that at a chosen position of the crotch part, basically along a cross-directional line over the upper surface of the top sheet, should at least x % of the surface include aperture or a perforation for allowing liquid to pass down into the liquid distribution layer and/or the absorbent structure. Thus, with reference to FIG. 4, this refers to D, being the diameter at the upper surface of the apertures. This value should not be confused with the open area, which would refer to d, the diameter of the effective aperture through the top sheet material.

By a “longitudinal” direction or “machine direction” is meant the direction along the length of the absorbent product, i.e., from the rear to the front of the product (or vice versa), and by “lateral” direction or “cross-direction” is meant the direction from side edge to side edge of the product, i.e., across the width of the product.

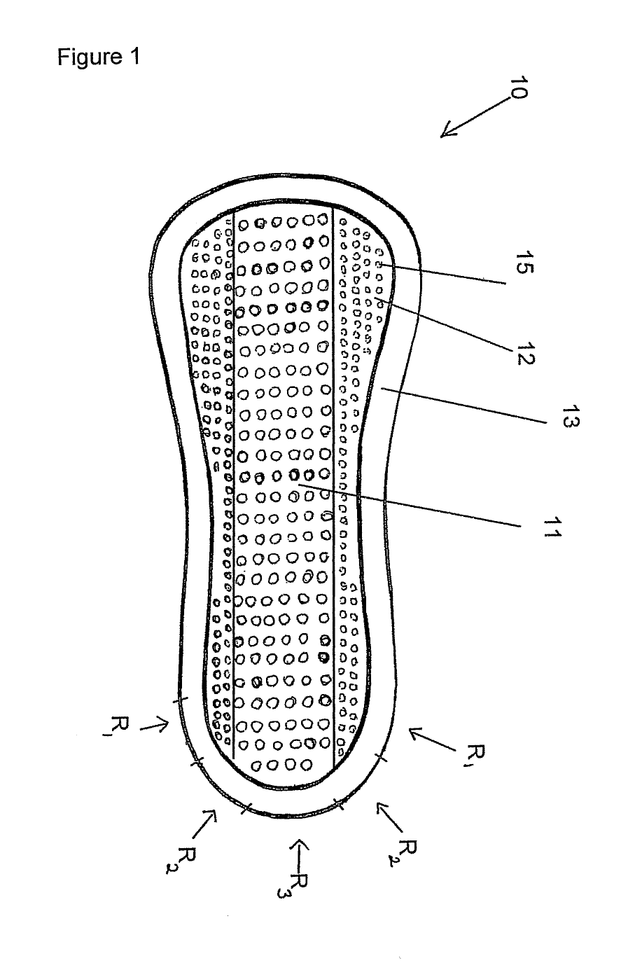

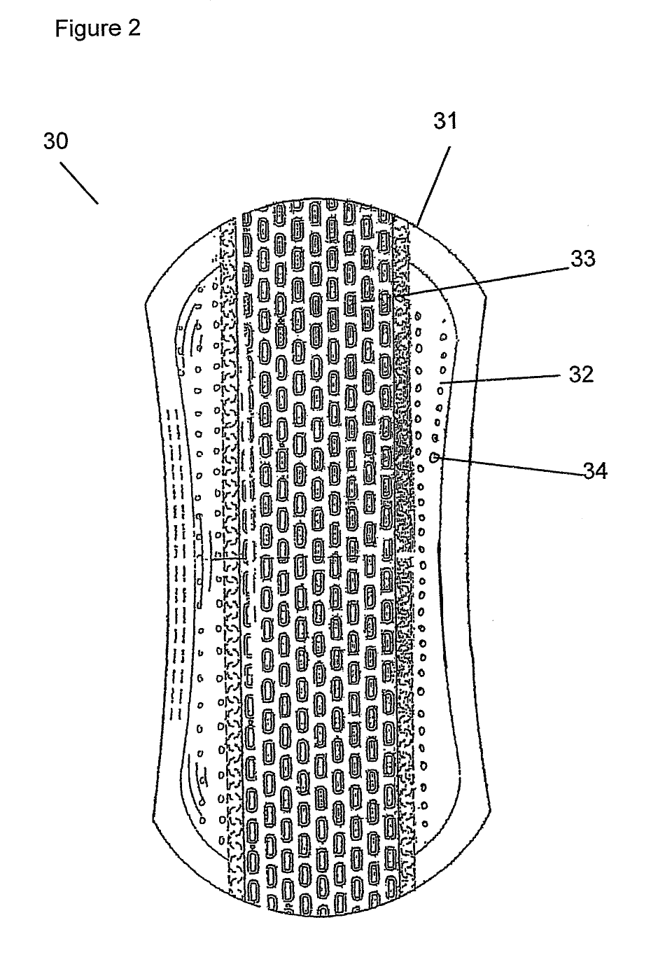

FIGS. 1 and 2 disclose absorbent products in the form of a panty liner (10) and a pad (30) of the invention. As can be seen in each figure, the product is equipped with an apertured top sheet (11, 33). At the longitudinal edges of the top sheet, textile-like edges (12, 32) are provided. Preferably, the textile-like edges are apertured (15, 34). Further, the textile-like edges can be equipped with an embossed pattern, in order to add function or for design purposes. At the periphery of the product, an edge sealing (13, 31) can be seen, which seals the various layers of the product. In a preferred embodiment, the edge sealing includes a pattern of discrete welding points, e.g., created by ultrasonic welding. Further (not shown), the top sheet preferably includes small perforations in addition to the larger apertures.

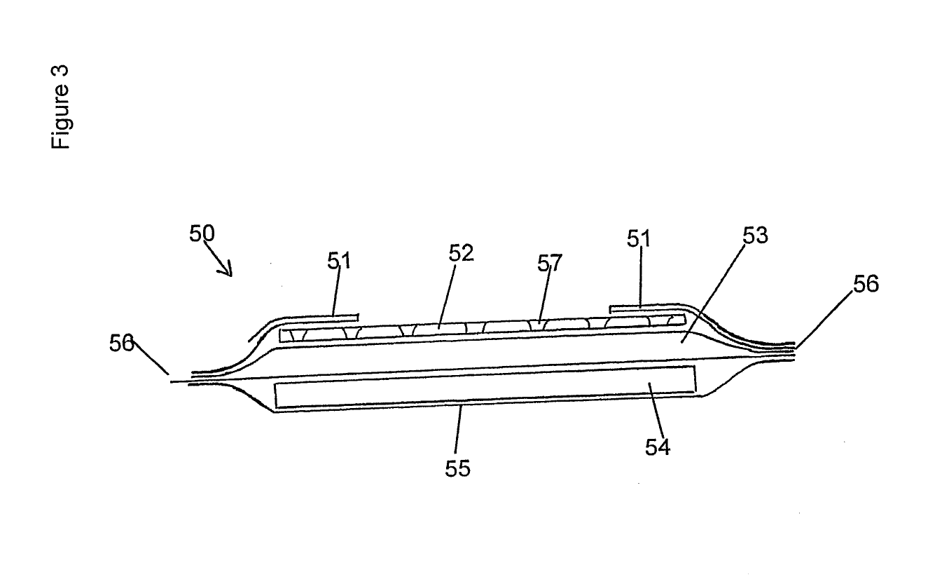

Turning to FIG. 3, a principal drawing of the various layers of the product (50) can be seen. Starting from the top (intended to be closest to the skin of the wearer at use) the textile-like edges (51) can be seen, which are positioned at the longitudinal edges of the top sheet (52). The textile-like edges can be fastened to the top sheet by way of embossing the textile-like edges, by ultrasonic bonding, by using an adhesive, such as a hotmelt, or a combination of fastening devices. The top sheet includes apertures (57), preferably three-dimensional and funnel-shaped. Further (not shown) the top sheet preferably includes small perforations (in addition to the larger apertures). Beneath the top sheet an optional liquid distribution layer (53) can be positioned. In a preferred embodiment the liquid distribution layer is a high loft layer. Beneath the liquid distribution layer, or directly beneath the top sheet (in case no liquid distribution layer is present), the absorbent structure (54) is positioned. For example, the absorbent structure is a pressure-bonded airlaid core comprising superabsorbent polymers. In a preferred embodiment, in order to provide a high absorption capacity, which is necessary for a product of this type, the absorbent structure includes about 50-60% super absorbent polymer. Beneath the absorbent structure, a back sheet (55) is provided. For example, the back sheet is a plastic film. Preferably, the plastic film is breathable. At the outside of the back sheet a release paper is positioned which is fastened to the back sheet, e.g., by glue strings or by any other conventional devices. An edge sealing (56), binding the textile edges, e.g., by way of ultra-sonic welding, the liquid distribution layer and the back sheet at the longitudinal edges can also be seen.

FIG. 4 shows schematically the three-dimensional structure and the apertures (43) of the top sheet (41) according to the invention. The distribution layer (42) is shown beneath the top sheet. The apertures have an upper diameter D and a lower diameter d. Typically D>d so that the holes are funnel-shaped. Liquid that comes in contact with the top sheet will flow into the apertures from the upper diameter to the lower diameter and further into the liquid distribution layer.

The material of the inlet zone (between the textile edges) has larger apertures (compared to the textile edges) that preferably are three-dimensionally funnel-shaped, so that the inlet zone is optimised for the function (inlet, dryness etc).

To the contrary, the textile edges are optimised for comfort (softness, airiness, etc.). Therefore, a two-dimensional material is more suitable to use for this part of the product. Also, a better visual appearance is obtained by using different materials for the inlet zone and the textile edges, respectively.

The apertures of the top sheet can be circular or oval shaped, having an elongation in the machine direction (normally the machine direction is the longitudinal direction of the article). The apertures can be arranged in rows that extend in the cross-direction, in the machine-direction or diagonally. Also the holes can be arranged randomly over the surface of the top sheet. Further, the holes can have varying sizes and shapes, e.g., some holes can have a symmetric shape and other holes can have an asymmetric shape.

The top sheet can have an aperture size of 1.6 to 3.2 mm in the machine direction (longitudinally) and 0.9 to 2.3 mm in the cross direction (laterally). The aperture size can be from 1.0 to 6.0 mm in diameter.

The apertures of the textile edge can be oval-shaped, slightly elongated in the machine direction. The holes can be round/circular, or oval in machine direction or cross direction. The density of apertures in the textile edges will normally be in the interval from 4-250/cm², preferably from 50-120/cm². Further, the apertures of the textile edges can be positioned in one or more than one longitudinal or lateral rows, or they can be positioned in a longitudinally extended row. Further, the apertures can have varying sizes and forms. They can also be concentrated (zoned) to one or more zones of the textile edge, such as close to the crotch area or the expected wetting point, so that the function of the apertures is concentrated to positions where it is especially desired.

The open area of the top sheet can be in the interval from 2 to 70%, preferably from 5-50%, more preferably from 10-20%, most preferably about 14%.

The present invention is mainly directed to absorbent products such as an incontinence protection, a sanitary napkin or a panty liner. Each product includes a rear part, a front part and a crotch part positioned there between.

The product can be hour-glass shaped (wherein the rear part and front part essentially equally wide measured in the cross-direction, and the crotch part is less wide than the front or rear part) or it can be asymmetrically shaped or it can have any other shape that is appropriate for products of this type. By asymmetrical can be meant that the product is least wide in the crotch part of the product (measured in cross-direction) and it is wider in the front part than in the rear part (measured at the widest position of the rear and front part respectively in cross-direction). For instance, the width in the crotch part of the product can be from 4-7 cm, especially from 4.5 to 5 cm, for example about 4.7 cm. The width at the widest position of the front part can be from 5 to 8 cm, especially from 6-7 cm, for example about 6.5 cm. The width at the widest position of the rear part can be from 4.5 to 6.5 cm, especially from 5 to 6 cm, for example 5.3 cm.

In one preferred embodiment, the product of the invention is a panty liner having a length in the interval from 13 to 22 cm, preferably of about 15.2 cm and a width at the least wide position of the crotch part, that is in the interval from 4 to 6.5 cm, preferably smaller than 5.0 cm. The top sheet material is a three-dimensional pre-perforated film which is equipped with further apertures, typically 7-9 apertures per row. At the edges of the top sheet, textile edges, preferably apertured, are provided that are composed of nonwoven material. A high loft layer is included as a liquid distribution layer. Further, the absorbent core is provided in the form of a roll-material including about 40-60% SAP. The edges of the product including the liquid distribution layer are sealed by way of ultra sonic bonding. The product further includes a back sheet, facing away from the wearer.

In another preferred embodiment, the invention refers to a panty liner including a carded nonwoven as a top sheet material. The carded material is preperforated and includes apertures. At the edges of the top sheet a textile edge, preferably apertured, is provided that is composed of nonwoven material. The liquid distribution layer is composed of an airlaid material and the absorbent core includes pulp and SAP. The edges of the product including the liquid distribution layer are sealed by way of ultra sonic bonding. The product further includes a back sheet, facing away from the wearer.

In still another preferred embodiment, the invention refers to a sanitary napkin including an apertured top sheet material. At the edges of the top sheet an apertured textile edge is provided that is composed of nonwoven material. Further, the sanitary napkin includes a liquid distribution layer and an absorbent core. The edges of the product including the liquid distribution layer are sealed by way of ultra sonic bonding. The product further includes a back sheet, facing away from the wearer.

In yet another preferred embodiment, the invention refers to an incontinence product or incontinence guard including a liquid pervious top sheet facing the wearer during use, optionally a liquid distribution layer underneath the top sheet, an absorbent core, and a liquid impermeable back sheet facing away from the wearer. Normally, the absorbent core includes a compressed mixed or layered structure of cellulosic fluff pulp and superabsorbent polymers, however further or other material combinations as disclosed below are fully possible. Also, instead of being provided in one layer, the absorbent core may include two separate layers, or more.

The shape of the products of the invention can be hour-glass shaped (i.e., being less wide in the central part compared to the front and rear parts, which are equally wide). Also, the products of the invention can be asymmetrically shaped, whereby the product is less wide in the central part compared to the front and rear parts, and whereby the rear part is less wide than the front part. Other shapes are also fully possible for the products of the invention.

The textile-like edges are preferably made by a soft, skin-friendly material, such as a nonwoven, so that any chafing against the skin of the wearer is reduced as much as possible. For example, the material of the textile-like edges can be a carded nonwoven. Preferably, the textile-like edges are apertured.

The liquid-permeable top sheet is preferably made of a material showing properties like dryness and softness at use of the absorbent product, as this sheet lies against the body of the wearer. It is desired, that the sheet has a soft and textile-like surface, which remains dry also at repeated wettings. The top sheet may for example be composed of nonwoven material with a soft and smooth surface, such as, for example, a spunbond made of polypropylene fibers. In order to keep the surface closest to the skin of the wearer dry, a hydrophobic nonwoven-material may be used, which has holes, so that openings are formed in the material, which openings are greater than the cavities between the fibers of the material. In this way, fluid may be led down through the holed openings in the top sheet to the underlying absorption core. Other examples of material in the top sheet may for example be holed plastic films, such as, for example, a holed polyethylene film. The top sheet may be connected to the underlying back sheet and to the absorption core by, for example, glue, ultra-sonic bonding or through some kind of thermal bonding. Preferably, the top sheet is an apertured nonwoven, having an aperture density of 3-15, preferably 6-12 and more preferably 7-9 apertures/cm².

It is desirable that the caliper value of the top sheet material is as high as possible, since a high caliper value has a beneficial effect on the acquisition. However, in order to not affect the softness of the material negatively, a balance needs to be obtained, and thus a caliper value in the interval from 1.3 to 1.7 mm, preferably about 1.5 mm, has shown to be adequate.

Also, the top sheet material can be a three-dimensional laminate of nonwoven and plastic film. The nonwoven can be a carded thermobonded 100%-polypropylene based, hydrophilic material. The plastic film can be hydrophilic, pre-perforated (small holes), and made of a blend of polyethylene and polypropylene.

Also, the nonwoven part of the top sheet material can be a spunbond nonwoven, an air-thru bonded nonwoven, a spunlaced (hydroentangled) nonwoven, a meltblown nonwoven, or a combination of these. Raw material can be polypropylene (PP), polyethylene (PE), polyester (PET), polyamide (PA), or a combination of these. If there is a combination, there can be a mixture of fibers from different polymers, but each fiber can also include different polymers (for example, bicomponent fibers PP/PE or copolymer PP/PE). The textile edge nonwoven can also include a percentage of natural fibers, such as pulp or viscose. The nonwoven can be hydrophilic, permanent hydrophilic or hydrophobic. The nonwoven can have a basis weight in the interval from 7 to 50 gsm.

The plastic film can also be made of PE or PP, PET, PLA or starch (or any other thermoplastic polymer), or a blend or copolymer of the polymers mentioned.

The perforated top sheet can also be made of a single layer material, such as a nonwoven or film (as described above).

The liquid-impermeable back sheet includes a flexible material, preferably a thin plastic film of PE (polyethylene), PP (polypropylene), a polyester, or some other kind of suitable material, such as a hydrophobic nonwoven-layer or a laminate of a thin film and a nonwoven material. These types of laminates are often used in order to achieve a soft and a textile-like surface of the back sheet. In order to accomplish an airier and comfortable product it is also possible to use breathable back sheets, which prevents fluid from coming out of the absorbent product, but that allows moisture to be ventilated. These breathable back sheets may be composed of single material layers, or of laminates of, for example, blown or moulded polyethylene films, which have been laminated with, for example, a nonwoven layer of spunbond or of spunbond-meltblown-spunbond (SMS).

The absorbent structure is typically built up by one or more layers of cellulose fibers, for example cellulose fluff pulp. Other materials, which may be used, are for example absorbing nonwoven material, foam material, synthetic fiber materials or peat. In addition to cellulose fibers or other absorbing materials, the absorbent structure may also include superabsorbent material, so called SAP (super absorbent polymers), that is material in the form of fibers, particles, granula, film or the like, which material has the ability to absorb fluid corresponding to several times the weight of the superabsorbent material. The superabsorbent material binds the fluid and forms a fluid-containing gel. Moreover, the absorbent structure may include binders, form-stabilizing components or the like. The absorbent structure may be chemically or physically treated in order to change the absorption properties. For instance, it is possible to provide an absorbent layer with compressed regions and/or being compressed in the entire layer(s) in order to control the fluid flow in the absorbent body. It is also possible to enclose the absorbent layer(s) in an envelope of, for example, tissue material. For example, the absorbent structure is an airlaid, pressure-bonded structure including 30-80%, preferably about 40-60%, more preferably 50-60% SAP.

Typically, the absorbent structure has in its longitudinal direction an outstretched form, and may for example be essentially rectangular, T-shaped or hourglass-shaped. An hourglass-shaped absorbent body is wider in the front and rear parts than in the crotch part, in order to provide an efficient fluid absorption simultaneously as the design facilitates the product to form and to close around the user, thereby giving a better fit around the legs. In a preferred embodiment of the present invention, the absorbent structure has essentially straight and parallel longitudinal edges. The rear and front edges are rounded in order to fit the shape of the product. Preferably, the rear edge is rounded in the form of at least three different radii. The front edge can also be rounded in at least three different radii.

Also, the absorbent structure can be provided in the form of a roll-material, whereby the material typically is provided with a high compression, or the structure can be formed by way of air-laying technique.

In yet another embodiment, the absorbent structure is equipped with a wicking layer, which wicking layer has the purpose to spread fluid towards the front part of the absorbent structure. Moreover, the wicking layer does not necessarily need to cover the whole absorbent structure, but should preferably cover at least the part of the absorbent structure being in the front part of the casing, more preferably the part being in the front and crotch parts of the casing, and most preferably the entire absorbent structure.

The wicking layer is of a moisture permeable material, preferably tissue paper or a hydrophilic non-woven, and functions to disperse the fluid, i.e. urine, passing through the liquid permeable top sheet, preferably in a direction towards the front part of the diaper. The wicking layer includes small capillaries directing the fluid towards smaller capillaries, due to capillary forces.

Additional liquid distribution layers may be used in the product of the invention, preferably between the absorbent structure and the top sheet. For instance, additional layers improving the properties may be used, such as a transfer layer or various types of fluid-spreading material layers or inserts, so called waddings or high-loft layers. Typically, the liquid distribution layer is a porous, resilient, relatively thick material layer, for example in the form of a fibrous high loft layer, a carded fibrous web, a tow material or other type of bulky and resilient fibrous material having a high momentaneous liquid receiving capacity and which can temporarily store liquid before it is absorbed by the underlying absorbent core. Also, the liquid distribution layer may be in the form of a porous foam material. Also, it may include two or more material layers. In a preferred embodiment, the liquid distribution layer extends to the side edges of the product, i.e., it has basically the form of the top sheet or the top sheet including the textile edges. Hereby, advantages with regard to liquid distribution, edge sealing etc, as discussed above, can be achieved. However, the liquid distribution layer can also be designed so that it does not extend into the edge sealings. The transfer layer can be an airlaid layer and it can include SAP.

Further, the product of the invention is equipped with a release paper that covers the outer side of the back sheet of the product. The release paper is fastened to the back sheet by way of glue strings.

At the periphery of the product, with a width of about 0.5 cm, an edge sealing is provided in order to secure the top sheet, the back sheet and optionally the liquid distribution layer to each other. The edge sealing can be made by way of adhering the layers to each other, by way of embossment, by way of heat welding, by way of ultra sonic bonding, or a combination of these methods. Preferably, ultra-sonic bonding is used. Also, the sealing can be made as a continuous sealing or as discrete point weldings. Preferably, the sealing is made as point weldings, since this provides some advantages with regard to, e.g., hydrophobic character of the welding points as discussed above. Typically, the welding points are at a distance of about 0.1-0.5 mm from each other preferably about 0.2 mm. For performing the ultra-sonic bonding, an ultra-sonic bonding equipment is used. When the ultra-sonic bonding is performed, a welding pattern is formed, which welding pattern can have a form that is visually attractive and/or that have technical advantages, such as an improved comfort for the wearer or effects with regard to liquid distribution or improved softness and/or dryness.

In order to prevent fluid to leak out, the absorbent product on the side that is facing the wearer may also be equipped with inner fluid barriers, which are attached in connection to the longitudinal edges. Preferably, the inner barriers are made of an essentially liquid-impermeable material, such as for example, a hydrophobic nonwoven or a plastic film, and are formed as a longitudinal path with a first edge being connected to the absorbent product and a second free edge, which is adapted for being in close contact with the user at use of the absorbent product. The second edge is equipped with one or more elastic elements, preferably an elastic thread, which in contracted state contracts the free edge, whereby an upstanding barrier is formed. The inner barrier may be designed as a strip of a single sheet, wherein the free edge is turned down in order to enclose the elastic element to prevent direct contact of the elastic thread to the user. Alternatively, the barrier may be formed of two combined layers, whereby the elastic thread is attached to the edge of the free end between the two layers. In this case, the inner layer of the barrier may be composed of an elongation of the top sheet and the outer layer of an essentially liquid-impermeable material, or the inner and outer layers of the barrier may be composed of one single material strip, which is folded around the elastic thread. Further, the elastics can be provided in the form of foam, e.g., in a band or thread, or the elastics can be provided in any other conventional way.

Further, the product of the invention may be equipped with wings. Also products equipped with other layers than described in this disclosure are also included in the scope of the invention.

### EXAMPLES

In all the attached examples, properties were compared between a panty-liner product according to one embodiment of the invention (here called “Tena”) and various conventional panty-liner products (see table 1 and 2 for data for the compared products). The compared products have structures that are similar to Tena. The “Tena-product” has an asymmetric shape (see FIG. 1 for a principal drawing), the top sheet is a perforated laminate between a nonwoven material and a plastic film having a basis weight of about 40 gsm having apertured top sheet that also includes small perforations. Further, the product includes textile edges, a nonwoven high loft layer (basis weight of about 50 gsm) as liquid distribution layer, an absorbent core of cellulose fibers and SAP (about 50-60%) and a breathable back sheet. The “Tena” product is a small panty liner product, and thus the experimental values should be interpreted with regard to a small panty liner. However, since the results that are shown for the Tena product can be regarded as an effect of using quick inlet materials (top sheet having large apertures, textile edges having apertures, and an absorbent core having a large amount of SAP (about 50-60%)), the analogous results can be expected for other product types that are formed in a similar way by the same principles, but being longer, heavier and thicker.

TABLE 1

| | | | Length |
|---|---|---|---|
| Product | Weight (g) | Thickness (mm) | (mm) |
| Tena | 3.4 | 2.9 | 152 |
| Alldays Normal | 2.8 | 2.9 | 150 |
| Always pantiliners Regular (US) | 3.1 | 2.6 | 230 |
| Alldays Small | 1.7 | 2.8 | 142 |
| Libresse Normal | 2.9 | 2.8 | 150 |
| Carefree Original | 2.4 | 3.1 | 160 |
| Kotex Normal | 2.1 | 2.6 | 152 |
| Poise light liners | 3.3 | 2.7 | 165 |
| Alldays Extra Large | 3.9 | 3.0 | 177 |
| Always pantiliners Long (US) | 4.1 | 3.1 | 280 |
| Carefree Maxi Large | 3.0 | 3.0 | 180 |
| Lindor active mini Ultra | 3.8 | 2.3 | 165 |
| Libresse Large | 3.8 | 2.6 | 175 |

TABLE 2

| | | Carefree | Libresse | Kotex | Alldays | Alldays | Alldays |
|---|---|---|---|---|---|---|---|
| Construction | Tena | Original | Normal | Normal | Small | Normal | Extra Large |
| Surface | laminate | Thermobound | Carded | Nw | 3D-PE | 3D-PE | 3D-PE |
| | | PP- | nw | “Coform” | “cotton- | “cotton-like” | “cotton-like” |
| | | nw | | | like” | | |
| Drainage | High loft | Thermobound | Airlaid | no | Thermo- | Thermo- | Thermo- |
| | layer | bicomponent | | | bound PP- | bound PP- | bound PP- |
| | | PP/PE | | | nw | nw | nw |
| Absorption | Novathin, | Novathin, | Pulp and | Airlaid | Airlaid | 2-layer | 2-layer |
| material | SAP | airlaid | SAP | pulp- | with SAP- | airlaid with | airlaid with |
| | | with SAP | | system | fibers | SAP | SAP |
| SAP (% of | About 55 | — | About 8 | no | — | About 24 | About 31 |
| product) | | | | | | | |

### Example 1

### Acquisition

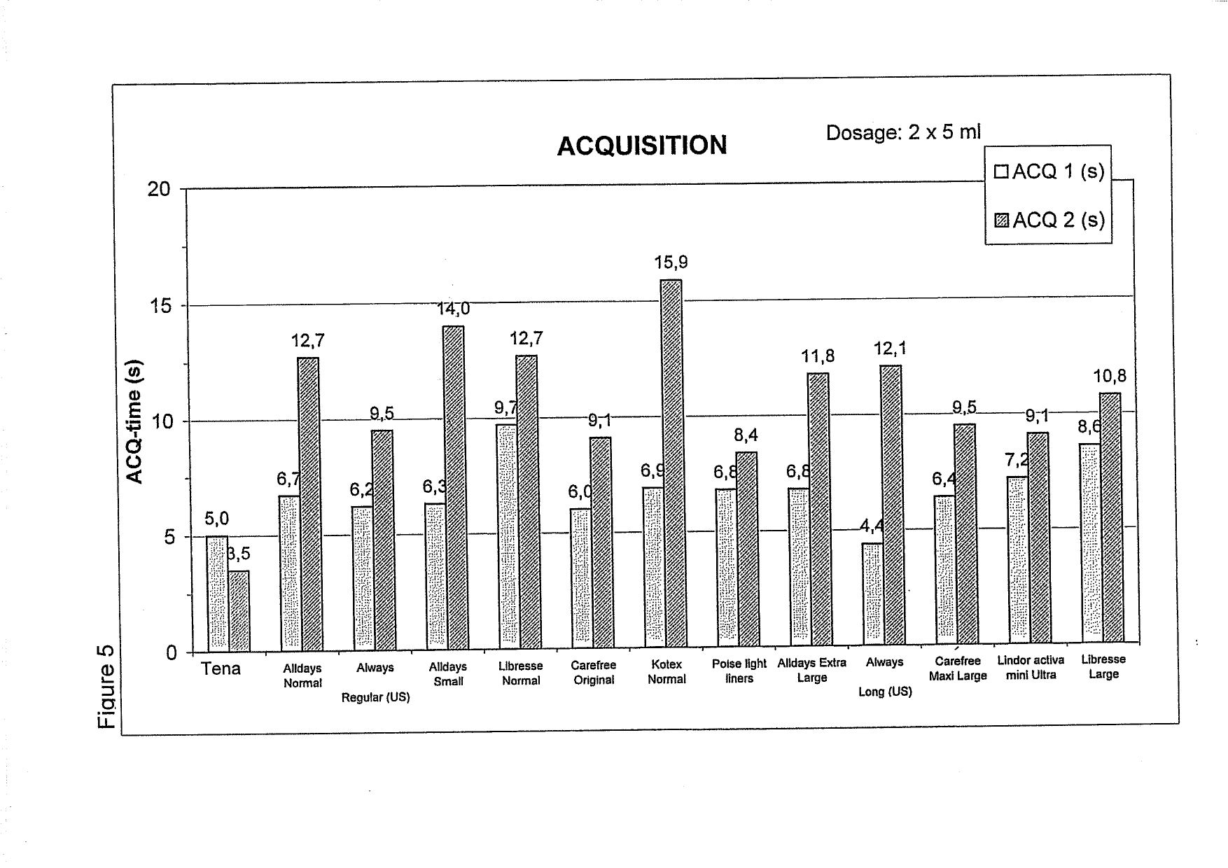

Acquisition properties (acquisition time) were tested (test method no 17-28-14) by exposing the various products for a first inlet of 5 ml synthetic urine (0.9% NaCl in water) followed by a second inlet of 5 ml synthetic urine with an interval of 10 min. The results can be seen in FIG. 5, showing that Tena is significantly faster than almost all the other products for the first inlet, with exception to Always pantiliners Long, and significantly faster than all other products for the second inlet. This shows that the construction of Tena is very capable of quick acquisition of liquid (not the least of urine type), thereby reducing risk for any leakage.

### Example 2

### DORUP (Retention)

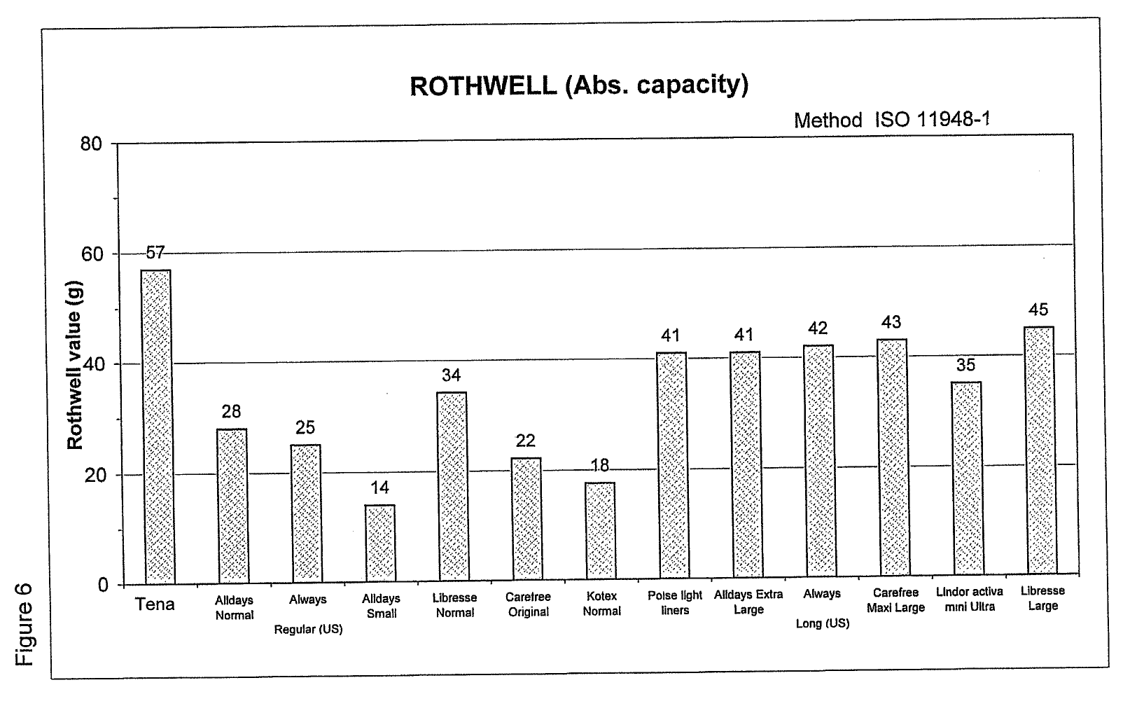

Retention properties were tested by exposing the various products for an inlet of 7 ml liquid (synthetic urine) (see FIG. 6). For more details concerning the DORUP (retention)-experiment, reference is made to U.S. Pat. No. 6,557,398, which is incorporated herein by reference. As can be seen, Tena is significantly drier than all compared products. This shows that the retention properties of Tena is superior to compared products.

### Example 3

### Rothwell (Absorption Capacity)

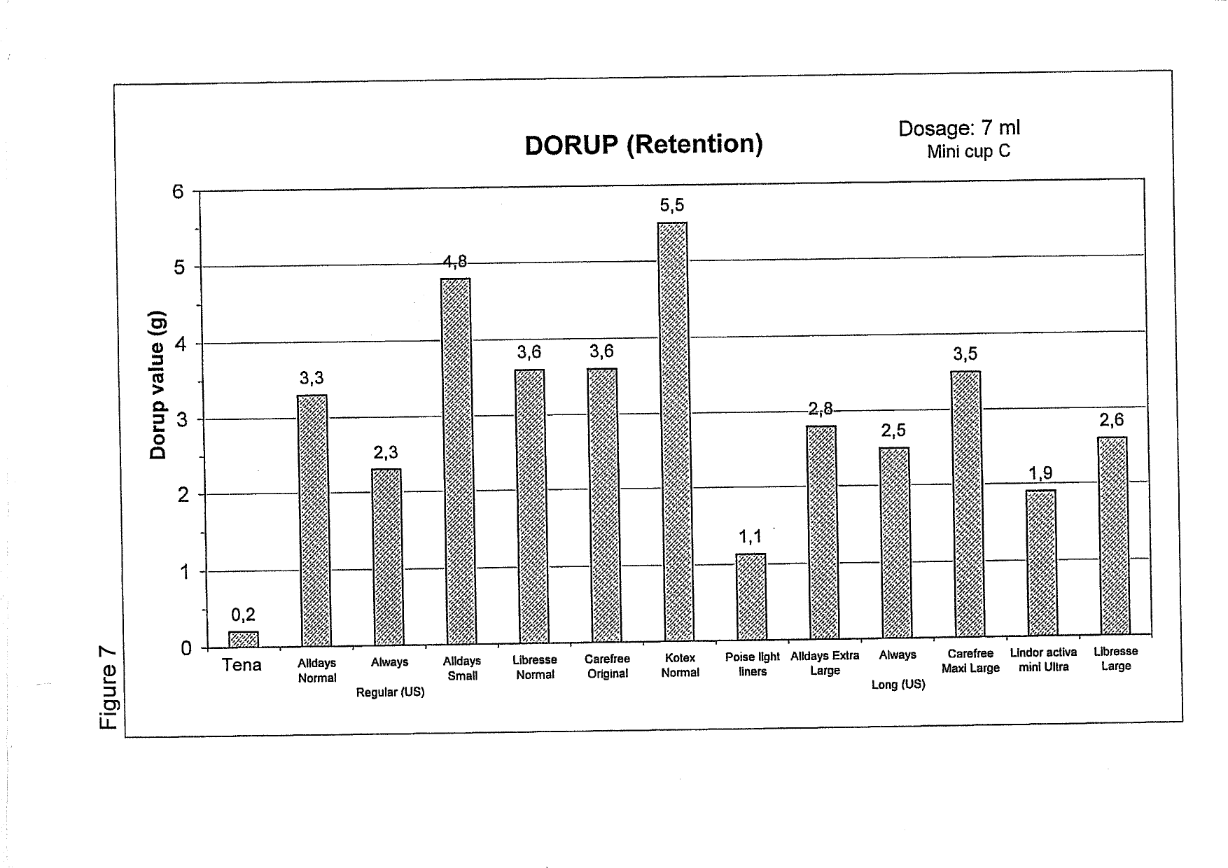

The Rothwell value (g) was tested (Method ISO 11948-1) (see FIG. 7). Tena was shown to have a significantly higher absorption capacity than all compared pantyliners.

1. An absorbent product chosen from an incontinence protection, a sanitary napkin and a panty liner, comprising:
a liquid permeable perforated top sheet facing the wearer during use,
a liquid impermeable back sheet facing away from the wearer during use, whereby longitudinally extending textile-like edges are positioned on both longitudinal sides of the top sheet, so that a central part of the top sheet is not covered by the textile-like edges,
wherein a longitudinal diameter of the apertures of the top sheet is in the interval from 0.1 to 3.2 mm, wherein at least 20% of the width of the upper surface of the top sheet of the crotch part is apertured or perforated or both, and wherein the textile-like edges are apertured.

2. The absorbent product according to claim 1, wherein the top sheet is three-dimensionally formed, and wherein each aperture has a longitudinal upper diameter (D) and a longitudinal lower diameter (d), wherein D is positioned closer to the top surface of the product, whereby D≧d.

3. The absorbent product according to claim 2, wherein D>d so that the apertures of the top sheet are funnel-shaped.

4. The absorbent product according to claim 1, wherein a density of the apertures in the central part of the top sheet is in the interval from 3-15 apertures/cm².

5. The absorbent product according to claim 1, wherein the top sheet further comprises perforations that are positioned between the apertures of the top sheet.

6. The absorbent product according to claim 1, wherein the perforations of the top sheet material have a diameter in the interval from 0.05 to 1 mm.

7. The absorbent product according to claim 1, wherein a density of perforations in the top sheet is in the interval from 20-500 perforations/cm².

8. The absorbent product according to claim 1, wherein the top sheet has a caliper value in the interval from 1.3 to 1.7 mm.

9. The absorbent product according to claim 1, wherein a width of the textile edges is in the interval from 10-30%, of a total product width at a least wide position of the crotch part of the product.

10. The absorbent product according to claim 1, whereby the ratio between the diameter of the apertures of the top sheet and the diameter of the apertures of the textile-like edges is at least 2.

11. The absorbent product according to claim 1, whereby the ratio between the open area of the top sheet and the open area of the textile-like edges is at least 2.

12. The absorbent product according to claim 1, wherein the product has a longitudinal length that is in the interval from 13-22 cm, and a lateral width, at the least wide position of the crotch part, that is in the interval from 4 to 6.5 cm.

13. The absorbent product according to claim 1, wherein the product has an acquisition time for a first inlet of 5 ml synthetic urine that is below 6 s, and an acquisition time for a second inlet of 5 ml synthetic urine that is below 8 s.

14. The absorbent product according to claim 1, wherein the product has a DORUP (retention) value for a dosage of 7 ml synthetic urine that is below 1 g.

15. The absorbent product according to claim 1, wherein the product has a Rothwell (Absorption capacity) value of more than 45 g.

16. The absorbent product according to claim 1, wherein the longitudinal diameter of the apertures of the top sheet is in the interval from 0.5 to 3.2 mm, wherein at least 50% of the width of the upper surface of the top sheet of the crotch part is apertured or perforated, and wherein the textile-like edges are apertured.

17. The absorbent product according to claim 1, wherein the longitudinal diameter of the apertures of the top sheet is in the interval from 1.6 to 2.4 mm, wherein at least 70% of the width of the upper surface of the top sheet of the crotch part is apertured or perforated or both.

18. The absorbent product according to claim 4, wherein the density of the apertures in the central part of the top sheet is in the interval from 7-9 apertures/cm².

19. The absorbent product according to claim 6, wherein the perforations of the top sheet material have a diameter in the interval from 0.1 to 0.4 mm.

20. The absorbent product according to claim 7, wherein the density of perforations in the top sheet is in the interval from 70-250 perforations/cm².

21. The absorbent product according to claim 7, wherein the density of perforations in the top sheet is in the interval from 120-170 perforations/cm².

22. The absorbent product according to claim 9, wherein the width of the textile edges is in the interval from 18-22%, of the total product width at the least wide position of the crotch part of the product.

23. The absorbent product according to claim 12, wherein the longitudinal length is smaller than 15 cm, and the lateral width, at the least wide position of the crotch part, is smaller than 5 cm.

24. The absorbent product according to claim 13, wherein the acquisition time for the first inlet of 5 ml synthetic urine is-equal to or less than 5 s, and the acquisition time for the second inlet of 5 ml synthetic urine is equal to or below 4 s.

25. The absorbent product according to claim 14, wherein the DORUP (retention) value for a dosage of 7 ml synthetic urine that is below 0.5 g.

26. The absorbent product according to claim 14, wherein the DORUP (retention) value for a dosage of 7 ml synthetic urine that is below 0.2 g.

27. The absorbent product according to claim 15, wherein the Rothwell (Absorption capacity) value is more than 55 g.

28. An absorbent product, comprising:
a liquid impermeable perforated top sheet facing the wearer during use;
a liquid impermeable back sheet facing away from the wearer during use; and
longitudinally extending textile-like edges posited along first and second longitudinal edges of the top sheet;
wherein a longitudinal diameter of apertures of the top sheet are sized between approximately 0.1 to 3.2 mm, at least 20% of a width of the upper surface of the top sheet is apertured or perforated or both, and the textile-like edges are apertured.
